(19) United States
(12) Patent Application Publication
(10) Pub. No.: US 2008/0207752 A1
(43) Pub. Date: Aug. 28, 2008

(54) Poly (epsilon-caprolactone-co-methylmethacrylate), method for preparing the same using super critical fluid, and method for preparing therapeutic agent for skin diseases such as acne, atopy and athlete's foot comprising the same

(76) Inventors: Moon Sam Shin, Seoul (KR); Hwayong Kim, Seoul (KR)

Correspondence Address: GREER, BURNS & CRAIN, LTD., Suite 2500, 300 South Wacker Drive, Chicago, IL, 60606, US

(21) Appl. No.: 11/711,323

(22) Filed: Feb. 27, 2007

(51) Int. Cl. A61K 31/22; A61P 17/00; C07C 69/22

(52) U.S. Cl. 514/546; 560/129

(57) ABSTRACT

A biodegradable polymer comprising a spherical poly (ε-caprolactone-co-methylmethacrylate) used in a therapeutic agent for skin diseases and a method for preparing the same, and a method for preparing the therapeutic agent for skin diseases having the particle size of 50 to 100 nm and a spherical shape comprising the poly (ε-caprolactone-co-methylmethacrylate) and salicylic acid.

The present invention relates to a novel poly(ε-caprolactone-co-methylmethacrylate), a method for preparing the same using supercritical fluid, and a method for preparing a therapeutic agent for skin diseases such as acne, atopy and athlete's foot comprising the poly(ε-caprolactone-co-methylmethacrylate) and salicylic acid as active ingredients.

### BACKGROUND OF THE INVENTION

1. Field of the Invention

The present invention relates to a poly(ε-caprolactone-co-methylmethacrylate), a method for preparing the same using supercritical fluid, and a method for preparing a therapeutic agent for skin diseases. More specifically, the invention relates to a novel poly(ε-caprolactone-co-methylmethacrylate), a method for preparing the same using supercritical fluid, and a method for preparing a therapeutic agent for skin diseases such as acne, atopy and athlete's foot comprising the poly(ε-caprolactone-co-methylmethacrylate) and salicylic acid as active ingredients.

2. Description of the Related Art

Salicylic acid is widely known as an active ingredient for skin diseases such as athlete's foot, atopy, and acne. Accordingly, there have been made attempts to use salicylic acid as a therapeutic agent for skin diseases. However, in practice, the side effects by the residual organic solvent still give the limitation in using salicylic acid as a therapeutic agent for skin diseases. In order to solve the problems caused from the residual organic solvent, the present invention aims to use a supercritical fluid as a substitute for the organic solvents.

Methods for obtaining crystal particles or microparticles using a supercritical fluid as a solvent or an anti-solvent have been actively studied. A supercritical fluid is an incompressible fluid under a temperature and pressure above each of the critical points, which shows unique characteristics not shown in the conventional organic solvents. Specifically, since the supercritical fluid near a critical point has molecular association by a density fluctuation, it has excellent properties such as a high density close to that of a liquid, a low viscosity and a high diffusion coefficient close to those of a gas, respectively, and a very low surface tension, simultaneously. Since the density of a supercritical fluid can be continuously changed from a sparse state like an ideal gas to a highly dense state like a liquid, its physical properties at equilibrium (e.g., a solubility, and an entrainer effect), mass transfer properties (e.g., a viscosity, a diffusion coefficient and a thermal conductivity) and a molecular clustering state of the fluid can be regulated.

Recently, technology using a supercritical fluid is widely employed in various fields of the polymer industry including uses for polymerization solvent. Carbon dioxide, in particular, has a relatively low critical point and no toxicity. Moreover, it is incombustible and inexpensive since it is generated as by-product of various chemical reactions. Further, in case of reducing the pressure, it can be changed from a supercritical state to a gas state. Therefore, there is an advantage that polymers can be easily separated from carbon dioxide. As a result, in respect of the separation process, energy can be saved. In respect of the chemistry, carbon dioxide can not be further oxidized and is very stable. Moreover, hydrogen is not contained in its structure, whereby chain transfer is not presented. Accordingly, there is an advantage that a polymerization reaction is available through the various synthetic methods and routes. Many inventions to take advantages of such the unique properties of a supercritical fluid have recently been made in biodegradable polymer synthesis.

In addition, in order to improve the skin permeability, a therapeutic agent should have a particle size of 50 to 100 nanometers. A drug with a particle size more than 100 nm may cause reduction of the skin permeability, whereas the drug with a particle size less than 50 nm may permeate to dermis, even to increase the toxicity. Furthermore, the drugs prepared by the conventional supercritical process have a problem of poor skin permeability due to its particle size of 1 to 10 micron.

Meanwhile, a biodegradable polymer is incorporated with a therapeutic agent for skin diseases in order to control the release rate of a drug. The biodegradable polymer has to be formed in the spherical particle, which make its surface area maximize to improve skin permeability. At present, poly(ε-caprolactone) is widely used as a biodegradable polymer. Poly(ε-caprolactone) is prepared by free radical polymerization of 2-methylene-1,3 dioxepane (MDOP). However, it has low glass transition temperature, 213.15 K, whereby it is hard to form the spherical particles at normal temperature. Therefore, its surface area may be reduced to generate the problem that the skin permeability is poor.

### SUMMARY OF THE INVENTION

In order to solve the above-described problems, an object of the present invention is to provide a novel spherical poly(ε-caprolactone-co-methylmethacrylate) as a biodegradable polymer used in therapeutic agent for skin diseases, a method for preparing the same, and a method for preparing the therapeutic agent for skin diseases having the particle size of 50 to 100 nm and a spherical shape comprising the poly(ε-caprolactone-co-methylmethacrylate) and salicylic acid using a supercritical fluid.

### DESCRIPTION OF THE DRAWINGS

FIG. 1. Scanning Electron Microscopy’ picture of a poly(ε-caprolactone-co-methylmethacrylate)

### DESCRIPTION OF THE PREFERRED EMBODIMENTS

A poly(ε-caprolactone-co-methylmethacrylate) according to present invention is a copolymer produced by copolymerizing a ε-caprolactone monomer and a methylmethacrylate monomer, which have the following structures.

A ε-caprolactone monomer is

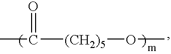

a methylmethacrylate monomer is

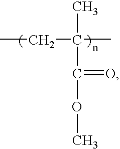

and a poly(ε-caprolactone-co-methylmethacrylate) according to the invention is

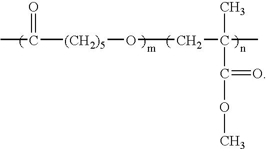

Wherein m is the number of ε-caprolactone monomers, n is the number of methylmethacrylate monomers. As usual, m and n are in the range of 10 to 1000, respectively.

The spherical poly(ε-caprolactone-co-methylmethacrylate) according to the invention is prepared as follows. ε-caprolactone and methylmethacrylate as monomers are injected into the reactor in the weight ratio of 1:1 to 1:1.5, and then 2,2-azobisisobutyronitrile (AIBN) as an initiator is injected in an amount of 0.1 to 1.0% by weight based on the total monomers. The mixture is subjected to polymerization under the condition of 50 to 300 bar and 283.15 K to 353.15 K for 1 to 168 hours.

The method for preparing a poly(ε-caprolactone-co-methylmethacrylate) will be described in detail as follows.

At first, a ε-caprolactone and a methylmethacrylate monomer are injected into a reactor in the weight ratio of 1:1 to 1:1.5. 2,2-azobisisobutyronitrile (AIBN) as an initiator is injected thereto in an amount of 0.1 to 1.0% by weight based on the total weight of monomers.

In the case where the proportion of ε-caprolactone and methylmethacrylate as a monomer is less than 1:1, the relative amount of ε-caprolactone is higher not to obtain the spherical biodegradable polymer due to low glass transition temperature. In the case where the proportion of ε-caprolactone and methylmethacrylate as a monomer is more than 1:1.5, the relative amount of methylmethacrylate is higher to make the affinity with a supercritical fluid poor. Therefore, aggregation is generated so that polymerization can not be facilitated. In the case where the proportion of 2,2-azobisisobutyronitrile (AIBN) as an initiator is less than 0.1% by weight based on the total monomers, the polymerization is not also initiated. In the case where the proportion of an initiator is more than 1.0% by weight, the reaction rate is too fast to obtain a copolymer having high molecular weight.

Next, the reaction conditions to facilitate a polymerization in the reactor are prepared as follows. The residual oxygen in the reactor is removed with nitrogen (2 bar) and then nitrogen is also removed with carbon dioxide (2 bar). While watching the liquid level through the transparent window, carbon dioxide is injected using a gas booster pump until the pressure reaches to 50 bar at normal temperature. Subsequently, the temperature in the reactor is increased to 283.15 K to 353.15 K using a thermostat and carbon dioxide is additionally injected into the reactor to make the pressure 50 to 300 bar. Under the above conditions, a ε-caprolactone monomer and a methylmethacrylate monomer are subjected to polymerization to prepare a poly(ε-caprolactone-co-methylmethacrylate). The polymerization was performed for 1 hour to maximum 168 hours, based on the point to reach each predetermined temperature.

Under the pressure of less than 50 bar, the mixture does not exist as homogeneous phase and phase separation occurs not to proceed a homogeneous reaction. Under the pressure of more than 300 bar, a high cost is required for a high pressure equipment to decrease operating economy. Under the temperature of less than 283.15 K, which is below its critical point, the mixture does not exist as homogeneous phase not to proceed a homogeneous reaction. Under the temperature of more than 353.15 K, the biodegradable polymer may be broken down due to high temperature. In the case where the polymerization is performed for 1 hour or less, a high molecular weight copolymer can not be obtained. In the case where the polymerization is performed for 168 hours or more, their viscosity is too high, due to their high molecular weight, to mix well, whereby the polymerization reaction can not be homogeneously performed.

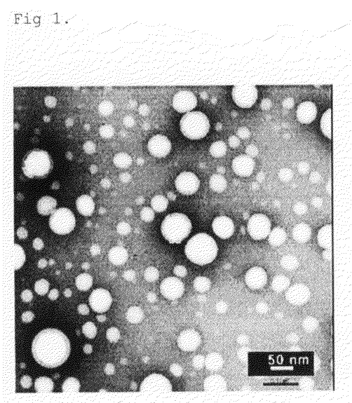

After completing the reaction for the predetermined time, the reactor was quickly cooled with ice. If the temperature of the reactor falls down to 283.15 K or lower, gas/liquid phase separation occurs and the solubility of the polymer comes to be very low to separate carbon dioxide slowly from the gas phase. Carbon dioxide was discharged out to obtain expanded solid polymers. After the solid polymer is washed with methanol, and dried under vacuum to obtain a spherical biodegradable poly(ε-caprolactone-co-methylmethacrylate) (see FIG. 1.) as a very fine white powdery solid.

From the result measured by using a differential scanning calorimeter (B. Wunderich, Thermal Analysis of Polymeric Materials, Springer, 2004), it is shown that glass transition temperature of the poly(ε-caprolactone-co-methylmethacrylate) as a biodegradable polymer in the invention is 303.15 K to 313.15 K, which is higher than that of poly(ε-caprolactone). From the result measured by using a gel permeation chromatography (J. F. Johnson; R. S. Porter, Analytical gel permeation chromatography, Interscience Publishers, 1968), it is shown that its average molecular weight is 5,000 to 50,000.

The poly(ε-caprolactone-co-methylmethacrylate) according to the invention, as well as salicylic acid, can be used as a raw material for therapeutic agent for skin diseases such as athlete's foot, atopy and acne. To maximize its efficacy, its particle has to be the size of 50 to 100 nm, and to be spherical shaped.

Hereinafter, the preparation method will be described in detail.

1) Step of Preparing Mixed Solution

At first, salicylic acid and a poly(ε-caprolactone-co-methylmethacrylate) as a biodegradable polymer are dissolved in an organic solvent to prepare a mixed solution.

The proportion of salicylic acid is preferably 0.0015 to 0.0018% by weight based on the total mixed solution. In the case where the proportion of the salicylic acid is less than 0.0015% by weight, the nucleation rate is so fast that the particle size gets to be over micron. In the case where the proportion of the salicylic acid is 0.0018% by weight or more, the nuclear growth rate is so fast that the plate shaped particles rather than spherical particles are obtained.

A biodegradable polymer is used for controlling the drug delivery rate. Therefore, the poly(ε-caprolactone-co-methylmethacrylate) prepared in the invention is preferably used as a spherical biodegradable polymer. A poly(ε-caprolactone-co-methylmethacrylate) has both of ε-caprolactone as a hydrophilic part and methylmethacrylate as a hydrophobic part in a molecule, which prevents particle aggregation generated upon nucleation and nuclear growth of the drug, that is, functions as a surfactant. Therefore, the drug in the form of the nanoparticles with a smaller size can be obtained.

The proportion of the biodegradable polymer is preferably 0.0013 to 0.0015% by weight based on the total mixed solution. In the case where the proportion of the biodegradable polymer is less than 0.0013% by weight, the nucleation rate is so fast that the particle size gets to be over micron. In the case where the proportion of the biodegradable polymer is 0.0015% by weight or more, the nuclear growth rate is so fast that the plate shaped particles rather than spherical particles are obtained. Another reason for using a poly(ε-caprolactone-co-methylmethacrylate) as a biodegradable polymer is that spherical particles cannot be formed with other biodegradable polymers such as poly(ε-caprolactone).

As an organic solvent, methanol, ethanol, or the like is used, but in the invention, ethanol having a low toxicity to a human is used.

2) Step of Forming Spherical Structure

As the supercritical fluid, supercritical carbon dioxide, supercritical dinitrogen monoxide, supercritical trifluoromethane, supercritical propane, supercritical ethylene, supercritical xenon, or the like is employable, but in a preferred example of the invention, supercritical carbon dioxide is used.

Carbon dioxide is injected into a stainless steel reactor, until the temperature and the pressure in the reactor reach 313.0 to 313.4 K and 149.5 to 151.3 bar, respectively. And pressure and heat are applied to the reactor. Therefore, the critical temperature and pressure of carbon dioxide reach to over 304.2K and 73.8 bar and carbon dioxide in the reactor is maintained at a supercritical state. The reactor is left to be equilibrated in a supercritical state. Upon the reactor being equilibrated, carbon dioxide is injected with the flow rate of 2.5 to 2.7 kg/hr and the mixed solution prepared in the preparation step is sprayed with the rate of 0.47 to 0.50 ml/min. In the case where the temperature and pressure are lower than those of the above condition, it is below its critical temperature and does not exist as homogeneous phase. In the case where the temperature and pressure are higher than those of the above condition, active ingredients may be deteriorated. Further, if the flow rate of carbon dioxide and the mixed solution is higher than those of the above condition, undesirable particles having the size of 100 nm or more are obtained. If the flow rate of carbon dioxide and the mixed solution is lower than those of the above condition, the plate shaped particles rather than spherical particles are obtained.

To check the exact amount of carbon dioxide injected, a syringe pump is used. A back pressure regulator maintains the constant pressure in the reactor by regulating the pressure of carbon dioxide introduced into the reactor. Further, a circular thermostat or an automatic temperature regulator is preferably used in order to maintain the constant temperature.

On the other hand, the mixed solution prepared in the preparation step is injected into the reactor at a constant rate using a small liquid pump capable of speed control. At this time, in order to prevent the nozzle from clogging, it is preferable to inject a small amount, for example, 3 to 4 ml, of a blank solvent, prior to injecting the solution. As the amount of the blank solvent injected increases, the time required for washing the reactor with the supercritical fluid becomes longer.

The solution injected is sprayed via a nozzle. The organic solvent in the sprayed solution is blended with the supercritical carbon dioxide rapidly, to form particles. In order to prevent the supercritical fluid in the reactor from being saturated during injecting the solution, a fresh supercritical fluid may further be injected into the reactor.

3) Step of Removing Organic Solvent

After the solution is completely sprayed into the reactor, a particle washing step is required by introducing a fresh supercritical fluid into the reactor to remove the organic solvent from the formed particles. In the above step, the supercritical fluid is injected at the constant rate, and discharged via an outlet at the same rate as the injection rate, which maintains the reactor pressure at 150 bars being allowed to obtain a particle size of 100 nm or less. At this time, a back pressure regulator is connected to the outlet to maintain the constant pressure in the reactor by regulating the discharging rate.

Double overlapping membrane filters having a pore size of 0.45 μm are installed at the outlet to hold the particles within the reactor. The washing step should be repeated until the residual solvent is completely removed, since if there is the residual solvent, the solvent re-dissolves the particles formed by precipitation, thus generating coagulation upon decreasing the temperature and the pressure for the recovery of the particles. The amount of the supercritical fluid for washing varies depending on the amount of the organic solvent used and the reactor size, preferably about 2,000 to 3,000 ml.

4) Step of Recovery

When the washing step is completed, the supply of the supercritical fluid into the reactor is stopped and the supercritical fluid is discharged. At this time, if the supercritical fluid is too rapidly discharged, the particles may be damaged. Accordingly, it is preferable to slowly discharge the supercritical fluid. After the supercritical fluid in the reactor is completely removed, the particles are recovered from the wall or bottom of the reactor.

### EXAMPLES

### (1) Synthesis of a poly(ε-caprolactone-co-methylmethacrylate)

Total 4 g of monomers which e-caprolactone (2 g) and methylmethacrylate (2 g) are mixed together, and 2 g of 2,2-azobisisobutyronitrile (AIBN) as an initiator are introduced into the 30-mL high pressure reactor. The residual oxygen in the reactor is removed with nitrogen (2 bar) and then nitrogen is also removed with carbon dioxide (2 bar). While watching the liquid level through the transparent window, carbon dioxide is injected using a gas booster pump until the pressure reaches to 50 bar at normal temperature. The temperature in the reactor is increased to predetermined temperature using a thermostat thereby the pressure increasing, and carbon dioxide is additionally injected thereto to make the pressure 255 bar at 333.15 K. At this time, the injected amount of carbon dioxide is 27.5 g. The polymerization was performed for 70 hours, based on the point to reach each predetermined temperature. After completing the reaction for the predetermined time, the reactor was quickly cooled with ice water. If the temperature of the reactor falls down to 283.15 K or lower, gas/liquid phase separation occurs and the solubility of the polymer comes to be very low to separate carbon dioxide slowly from the gas phase. At this time, to confirm whether the polymers are taken out or not, the presence of precipitate was observed in methanol filled in trap. Carbon dioxide was discharged out to obtain expanded solid polymers. After the solid polymer is washed with methanol, and dried under vacuum to obtain a spherical biodegradable poly(ε-caprolactone-co-methylmethacrylate) as a very fine white powdery solid.

### (2) Preparation of Therapeutic Agent for Skin Diseases

At first, pretreated salicylic acid is 50-100 micron. In Examples, the size of salicylic acid particles prepared in following manners was measured using a particle size analyzer. Spherical nanoparticles of which the particle size is in the range of 50 to 300 nm can be obtained according to the presence of a poly(ε-caprolactone-co-methylmethacrylate) as a biodegradable polymer and operating conditions in the reactor.

As shown in the following Table 1, the present Examples represent the operating conditions of the reactor in the step of formation of a spherical structure. The temperature and the pressure of the reactor must be each above 304.2 K and 73.8 bar. The operating parameters in the reactor are the temperature and the pressure in reactor, the amount of carbon dioxide as a supercritical fluid, and the amount of the mixed solution prepared in the preparation step. In Example 1, the mixed solution contains salicylic acid of 0.0016% by weight alone without a biodegradable polymer. In Example 2 to 8, the solution contains both of salicylic acid of 0.0016% by weight and the poly(ε-caprolactone-co-methylmethacrylate) of 0.0014% by weight as a biodegradable polymer. It is noted that the particle sizes are decreased to around 100 nm in the case of the solution containing the poly(ε-caprolactone-co-methylmethacrylate) as a biodegradable polymer. In Examples 1 and 3, the change was measured at 313K and 323K by adjusting the reactor temperature. As a result, if the temperature is increased, the particle size tends to be increased. In Examples 1, 4 and 5, if the reactor pressure was adjusted from 130 to 170 bar, particles with the optimal sizes can be obtained at a reactor pressure of 150 bar. In Examples 1, 6 and 7, if the flow rate of carbon dioxide was changed from 2.5 to 3.0 kg/hr, particles with the optimal sizes can be obtained in the flow rate of carbon dioxide of 2.5 to 2.7 kg/hr. In Examples 1 and 8, the flow rate of the solution was changed from 0.5 to 1.0 ml/min, and it was found that as the amount of the mixed solution is increased, the particle size is increased.

TABLE 1
Operating conditions and particle sizes according to
four operating parameters in Example 1 to 8

| | | | Flow rate of | | |
|---|---|---|---|---|---|
| | Reactor | Reactor | carbon | Flow rate of | Particle |
| | temperature | pressure | dioxide | solution | size |
| Example | (K) | (bar) | (kg/hr) | (ml/min) | (nm) |
| Example 1 | 313 | 150 | 2.5 | 0.5 | 589 |
| Example 2 | 313 | 150 | 2.5 | 0.5 | 86 |
| Example 3 | 323 | 150 | 2.5 | 0.5 | 135 |
| Example 4 | 313 | 130 | 2.5 | 0.5 | 120 |
| Example 5 | 313 | 170 | 2.5 | 0.5 | 103 |
| Example 6 | 313 | 150 | 2.7 | 0.5 | 73 |
| Example 7 | 313 | 150 | 3.0 | 0.5 | 149 |
| Example 8 | 313 | 150 | 2.5 | 1.0 | 200 |

The operating conditions of Example 2 and 6 are suitable for obtaining preferable nanoparticles of 50 to 100 nm. That is, the particle size could be significantly decreased by incorporating the poly(ε-caprolactone-co-methylmethacrylate) as a biodegradable polymer. The optimal condition is such that the temperature is 313 K, the pressure is 150 bar, the flow rate of carbon dioxide is 2.5 to 2.7 kg/hr and the flow rate of the solution is 0.5 ml/min.

From more specifically performed experiments, it was found that in order to obtain supercritical nanoparticles having a particle size of 50 to 100 nm, the proportions of salicylic acid as a drug and the poly(ε-caprolactone-co-methylmethacrylate) as a biodegradable polymer are 0.0016% by weight and 0.0014% by weight, respectively. It was also found that a desired range of the preferable sizes could be obtained, if the operating conditions are that the temperature is 313.0 to 313.4 K, the pressure is 149.5 to 151.3 bar, the flow rate of carbon dioxide is 2.5 to 2.7 kg/hr, and the flow rate of the solution is 0.47 to 0.50 ml/min. Under the operating conditions out of the above ranges for the temperature, the pressure, the flow rate of carbon dioxide and the flow rate of the solution, an unwanted particle size of more than 100 nm are obtained, thus it not being preferable.

According to the present invention, a poly(ε-caprolactone-co-methylmethacrylate) was prepared as a spherical biodegradable polymer, the drug release rate can be regulated by incorporating salicylic acid for acne treatment with the poly(ε-caprolactone-co-methylmethacrylate) as a biodegradable polymer, and supercritical nanoparticles having a particle size of 50 to 100 nm (Examples 2 and 6) can be obtained by controlling the conditions of the process, whereby the effective cross-sectional area is increased to improve skin permeability.

What is claimed is:

1. A poly(ε-caprolactone-co-methylmethacrylate) wherein its molecular weight is 5000 to 50000 and its main chain is the following repeating unit.

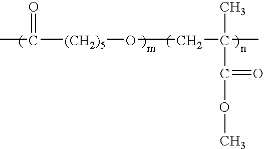

2. A method for preparing the poly(ε-caprolactone-co-methylmethacrylate) having the molecular weight of 5,000 to 50,000, wherein ε-caprolactone and methylmethacrylate as monomers are injected into the reactor in the weight ratio of 1:1 to 1:1.5, 2,2-azobisisobutyronitrile (AIBN) as an initiator is injected in an amount of 0.1 to 1.0% by weight based on the total monomers and the mixture is subjected to polymerization under the condition of 50 to 300 bar and 283.15 K to 353.15 K for 1 to 168 hours.

3. A method for preparing a therapeutic agent for skin diseases such as acne, atopy and athlete's foot comprising the poly(ε-caprolactone-co-methylmethacrylate), wherein the method comprises the steps of:
dissolving poly(ε-caprolactone-co-methylmethacrylate) and salicylic acid in an organic solvent to prepare a mixed solution;
spraying the mixed solution into a reactor charged with a supercritical fluid to prepare spherical nanoparticles;
introducing fresh carbon dioxide into the reactor to remove the organic solvent; and
recovering spherical nanoparticles after removal of the organic solvent, and wherein
in the step of preparing the solution, the proportion of poly(ε-caprolactone-co-methylmethacrylate) is 0.0013 to 0.0015% by weight, and the proportion of salicylic acid is 0.0015 to 0.0018% by weight, based on the total amount of the mixed solution, and ethanol is used as an organic solvent;
in the step of preparing spherical nanoparticles, carbon dioxide is used as a supercritical fluid, the temperature and the pressure in the reactor are 313.0 to 313.4 K, and 149.5 to 151.3 bar, respectively, and the flow rates of carbon dioxide and the mixed solution are 2.5 to 2.7 kg/hr, and 0.47 to 0.50 ml/min, respectively; and
in the step of removing the organic solvent, the pressure at the outlet of the reactor is maintained at 150 bar.
